(19) United States
(12) Patent Application Publication
(10) Pub. No.: US 2009/0069341 A1
(43) Pub. Date: Mar. 12, 2009

(54) INHIBITORS OF HSP90

(76) Inventors: Patrick Chene, Mulhouse (FR); Andreas Floersheimer, Dornach (CH); Pascal Furet, Thann (FR); Joseph Schoepfer, Riehen (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/658,365

(22) PCT Filed: Jul. 26, 2005
(86) PCT No.: PCT/EP2005/008118

§ 371 Date: Nov. 10, 2008

Related U.S. Application Data
(60) Provisional application No. 60/591,330, filed on Jul. 27, 2004.

(51) Int. Cl. A61K 31/497; C07D 235/12; A61K 31/4184; A61P 35/00; C07D 403/10

(52) U.S. Cl. 514/254.06; 548/304.4; 544/370; 514/395

(57) ABSTRACT

The invention relates to the use of benzoimidazolone compounds and salts thereof in the treatment of proliferative diseases and for the manufacture of pharmaceutical preparations for the treatment of said diseases, pharmaceutical preparations comprising benzoimidazolone compounds, novel benzoimidazolone compounds, and a process for the preparation of the novel benzoimidazolone compounds.

### SUMMARY OF THE INVENTION

The invention relates to methods of use of benzoimidazolone derivatives in the treatment of proliferative diseases, pharmaceutical preparations comprising benzoimidazolone derivatives for the treatment of said diseases, or for the manufacture of pharmaceutical compositions for use in the treatment of said diseases. The present invention also relates to novel benzoimidazolone derivatives, pharmaceutical preparations comprising these benzoimidazolone derivatives, processes for the manufacture of the novel benzoimidazolone derivatives and pharmaceutical preparations, and novel intermediate compound used in the manufacture of benzoimidazolone derivatives.

### BACKGROUND OF THE INVENTION

The Hsp90 family of chaperones is comprised of four known members: Hsp90α and Hsp90β both in the cytosol, grp94 in the endoplasmic reticulum and trap-1 in the mitochondria. Hsp90 is an abundant cellular chaperone required for the ATP-dependent refolding of denatured or “unfolded” proteins and for the conformational maturation of a variety of key proteins involved in the growth response of the cell to extracellular factors. These proteins, which are called client proteins, include the steroid receptors as well as various protein kinases. Hsp90 is essential for eukaryotic cell survival and is overexpressed in many tumors. Cancer cells seem to be sensitive to transient inhibition of Hsp90 ATPase activity suggesting that Hsp90 inhibitors could have a potential as new anticancer drugs. Each Hsp90 family member possesses a conserved ATP-binding site at its N-terminal domain, which is found in few other ATP-binding proteins. The weak ATPase activity of Hsp90 is stimulated upon its interaction with various co-chaperone proteins. Several natural compounds such as geldanamycin or radicicol bind at the ATP-binding site of Hsp90 inhibiting its ATPase activity. In cellular systems and in vivo, these drugs upon binding to Hsp90 prevent the folding of the client proteins, which are then degraded in the proteasome. 17-allylamino-17-demethoxygeldanamycin (17-AAG), a geldanamycin derivative, is currently in Phase I clinical trial at several institutions. Initial clinical experiences with 17-AAG have offered preliminary evidence that concentrations of the drug associated with activity in pre-clinical systems can be achieved in humans with tolerable toxicity, and provided early evidence of target modulation in at least certain surrogate and tumor compartments. The dose limiting toxicity of 17-AAG is hepatic. 17-AAG poor solubility makes it difficult to formulate/administer and its synthesis is difficult (it is generally obtained by fermentation). Therefore synthetic compounds with better physicochemical properties and maybe of higher specificity (17-AAG inhibits all these the four Hsp90 paralogs) are needed in clinic.

There is an ever-existing need to provide novel classes of compounds that can inhibit Hsp90 and therefore trigger apoptosis of proliferating cells.

We have now found that the benzoimidazolone residue can be also be used as template for the design of compounds which act as Hsp90 inhibitors.

### GENERAL DESCRIPTION OF THE INVENTION

The class of benzoimidazolone compounds described herein, especially novel compounds falling under this class, has surprisingly been found to have pharmaceutically advantageous properties, inter alia, as Hsp90 inhibitors.

### DETAILED DESCRIPTION OF THE INVENTION

The invention relates to use of benzoimidazolone compounds of the formula (I):

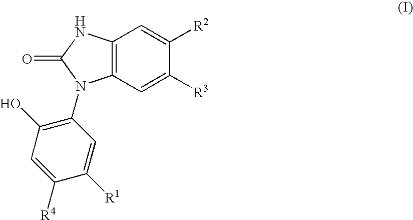

wherein:
R¹ is H, halo, substituted or unsubstituted lower alkyl;
R² is H, halo, substituted or unsubstituted lower alkyl, carboxy, COR⁵, SO₂R⁵, CX₂R⁵, CXHR⁵, CH₂R⁵, CHR⁵R⁶, C R⁵(R⁶)₂, or C(R⁵)₂R⁶;
R³ is H, substituted or unsubstituted lower alkyl, halo, —SO₂NH₂ or

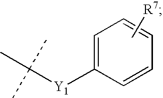

R⁴ is H or hydroxy;
R⁵ is lower alkyl; —(CH₂)ₙ—NR⁶₂; —YR⁶; —Y(CH₂)ₘ—NR⁶₂;

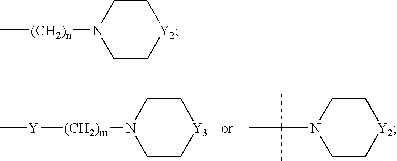

n is 1 or 2;
m is 2 or 3;
X is halo;
Y₁ is alkylene, O, S or N;
Y₂ and Y₃ are each independently methylene, O or NR′;
R⁶ is H, lower alkyl, cycloalkyl, heterocycl, fused cycloalkyl, fused heterocycl or NR⁹R¹⁰ together form a heterocyclic ring with the N atom, form a 3- to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl); cycloalkyl as defined above, especially C₃-C₆-cycloalkyl, lower alkanoyl (preferably as single amino substituent or in combination with another of the non-acyl moiety just mentioned) and benzoyl or phenyl-lower alkanoyl (preferably as single amino substituent or in combination with another of the non-acyl moiety just mentioned), cyano, cyano-lower alkyl, such as cyanomethyl, amidino, N-hydroxyamidino, amidino-lower alkyl, such as -methyl, or N-hydroxyamidino-lower alkyl, such as -methyl;
R⁷ is lower alkyl, halo, lower alkoxy, or —Y₁—(CH₂)ₚ—N(R⁸)(H);
p is 1-3;
R⁸ is H or lower alkyl;
or pharmaceutically acceptable salts thereof,
in the treatment of proliferative diseases, especially those dependent on Hsp90 activity, or for the manufacture of pharmaceutical compositions for use in the treatment of said diseases, methods of use of compounds of formula (I) in the treatment of said diseases, pharmaceutical preparations comprising compounds of formula (I) for the treatment of said diseases, compounds of formula (I) for use in the treatment of said diseases.

A preferred embodiment of the invention relates to benzoimidazolone compounds of the formula (IA):

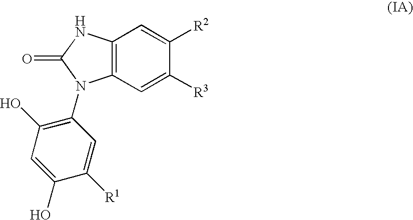

wherein:
R¹ is H, halo, substituted or unsubstituted lower alkyl;
R² is H, halo, substituted or unsubstituted lower alkyl, carboxy, COR⁵, SO₂R⁵, CX₂R⁵, CXHR⁵, CH₂R⁵, CHR⁵R⁶, C R⁵(R⁶)₂, C(R⁵)₂R⁶;
R³ is H, substituted or unsubstituted lower alkyl, halo, —SO₂NH₂ or

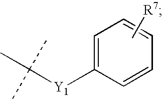

R⁵ is lower alkyl; —(CH₂)ₙ—NR⁶₂; —YR⁶; —Y(CH₂)ₘ—NR⁶₂;

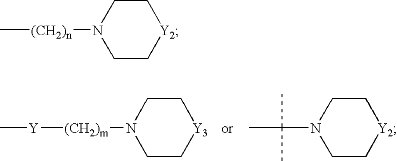

n is1 or 2;
m is 2 or 3;
X is halo;
Y₁ is alkylene, O, S or N;
Y₂ and Y₃ are each independently methylene, O or NR′;
R⁶ is H, lower alkyl, cycloalkyl, heterocycl, fused cycloalkyl, fused heterocycl or NR⁹R¹⁰ together form a heterocyclic ring with the N atom, form a 3- to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl); cycloalkyl as defined above, especially C₃-C₆ cycloalkyl, lower alkanoyl (preferably as single amino substituent or in combination with another of the non-acyl moiety just mentioned) and benzoyl or phenyl-lower alkanoyl (preferably as single amino substituent or in combination with another of the non-acyl moiety just mentioned), cyano, cyano-lower alkyl, such as cyanomethyl, amidino, N-hydroxyamidino, amidinolower alkyl, such as -methyl, or N-hydroxyamidino-lower alkyl, such as -methyl;
R⁷ is lower alkyl, halo, lower alkoxy, —Y₁—(CH₂)ₚ—N(R⁸)(H);
p is 1-3;
R⁷ is H or lower alkyl;
or pharmaceutically acceptable salts thereof,
and use of such compounds in the treatment of proliferative diseases, especially those dependent on Hsp90 activity, or for the manufacture of pharmaceutical compositions for use in the treatment of said diseases, methods of use of compounds of formula (IA) in the treatment of said diseases, pharmaceutical preparations comprising compounds of formula (IA) for the treatment of said diseases, compounds of formula (IA) for use in the treatment of said diseases.

The general terms used hereinbefore and hereinafter preferably have within the context of this disclosure the following meanings, unless otherwise indicated:

“Alkyl” includes lower-alkyl preferably alkyl with up to 10 carbon atoms, preferably from 1 to and including 5, and is linear or branched; preferably, lower alkyl is methyl, ethyl, propyl, such as n-propyl or isopropyl, n-butyl, isobutyl, sec-butyl, tert-butyl, straight or branched pentyl, straight or branched hexyl, straight or branched heptyl, straight or branched nonyl or straight or branched decyl. Preferably alkyl is C₁ to C₄-alkyl especially methyl, ethyl, propyl, 2-methyl propyl and t-butyl. The alkyl group may be unsubstituted or substituted with any of the substituents defined below, preferably halo, hydroxy, lower alkoxy (such as methoxy), phenyl, cycloalkyl (such as cyclopropyl), lower alkyl or substituted lower alkyl (such as diphenyl methyl).

Most preferably the alkyl group is a lower alkyl of 1-4 carbon atoms, preferably methyl, ethyl, propyl, butyl, isobutyl, tertbutyl, and isopropyl.

Most preferably the alkyl group is substituted with halo, cyclopropyl or substituted or unsubstituted phenyl.

“Alkylene” includes lower alkylene preferably alkylene with up to 10 carbon atoms, preferably from 1 to and including 5, most preferably methylene, ethylene or propylene.

“Aryl” is an aromatic radical having 6 to 14 carbon atoms, which is unsubstituted or substituted by one or more, preferably one or two substituents, wherein the substituents are as described below. Preferred “aryl” is phenyl which may be substituted with any of the substituents defined below, preferably lower alkyl (such as methyl); lower alkoxy (such as methoxy); hydroxy; or halo.

A “cycloalkyl” group means C₃ to C₁₀-cycloalkyl having 3 to 8 ring carbon atoms and may be, for example, cyclopropyl, cyclobutyl, cyclopentyl, cyclohexyl, cycloheptyl or cyclooctyl. Preferably, cycloalkyl is cyclopropyl. The cycloalkyl group may be unsubstituted or substituted with any of the substituents defined below.

“Halo” includes fluoro, chloro, bromo and iodio, with fluoro, chloro and bromo being most preferred.

A “heteroaryl” group is mono-, bi- or tri-cyclic, and comprises 3-24, preferably 4-16 ring atoms, wherein at least one or more, preferably one to four ring carbons are replaced by a heteroatom selected from O, N or S such as oxiranyl, azirinyl, 1,2-oxathiolanyl, imidazolyl, thienyl, furyl, tetrahydrofuryl, indolyl, azetidinyl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, pyranyol, thiazolyl, isothiazolyl, dithiazolyl, oxazolyl, isoxazolyl, pyridyl, pyrazinyl, pyrimidinyl, piperidyl, piperazinyl, pyridazinyl, morpholinyl, thiomorpholinyl, indolizinyl, isoindolyl, 3H-indolyl, indolyl, benzimidazolyl, benzothiazolyl and benzo[1,2,5]thiadiazolyl, thiacumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, β-carbolinyl, phenanthridinyl, acridinyl, perimidinyl, phenanthrolinyl, furazanyl, phenazinyl, phenothiazinyl, phenoxazinyl, chromenyl, isochromanyl and chromanyl, each of these radicals being unsubstituted or substituted by one to two radicals selected from the list described below.

“Heterocycl” radical refers to a heterocyclic ring containing 1-4 nitrogen, oxygen or sulfur atoms (e.g. piperazinyl, lower alkyl-piperazinyl, azetidinyl, pyrrolidinyl, piperidino, morpholinyl, imidazolinyl). Heterocyclyl is preferably a heterocyclic radical that is unsaturated, saturated or partially saturated in the bonding ring; has 3-24, more preferably 4-16 ring atoms, wherein at least in the ring bonding to the radical of the molecule of formula (I) of (IA) one or more, preferably 1-4, especially one or two carbon ring atoms are replaced by a heteroatom selected from the group consisting of nitrogen, oxygen and sulfur, the bonding ring preferably having 4-12, especially 4-7 ring atoms; heterocycl being unsubstituted or substituted by one or more, especially 1-4 substituents independently selected from the group consisting of the substituents defined above under “substituted”; especially being a heteroaryl radical selected from the group consisting of indoly, benzofuranyl, thienyl, pyridyl, imidazolinyl, morpholinyl, piperazinyl, piperidino, piperidyl, pyrrolidinyl and azetidinyl, with piperazinyl being especially preferred.

Any of the above defined aryl, alkyl, cycloalkyl, may be unsubstituted or independently substituted by up to four, preferably one, two or three substituents, selected from the group consisting of: halo (such as F, Cl or Br); hydroxy; lower alkyl (such as C₁-C₃ lower alkyl); lower alkyl which may be substituted with any of the substituents defined herein; lower alkenyl; lower alkynyl; lower alkanoyl; alkoxy (such as methoxy); aryl (such as phenyl or benzyl); substituted aryl (such as alkyl phenyl, alkoxy phenyl, amino alkoxy phenyl, alkyl amino alkoxy phenyl or dialkyl amino alkoxy phenyl); amino; mono- or disubstituted amino; amino alkyl (such as dimethylamino); acetyl amino; amino alkoxy (such as amino ethoxy); alkyl amino alkoxy; dialkyl amino alkoxy; alkoxy amino (such as ethoxyamine); nitro; cyano; cyano lower alkyl; carboxy; esterified carboxy (such as lower alkoxy carbonyl e.g. methoxy carbonyl); n-propoxy carbonyl or iso-propoxy carbonyl; alkanoyl; benzoyl; carbamoyl; N-mono- or N,N-disubstituted carbamoyl; carbamates; alkyl carbamic acid esters; amidino; guanidine; urea; ureido; mercapto; sulfo; lower alkylthio; sulfoamino; sulfonamide; benzosulfonamide; sulfonate; sulfanyl lower alkyl (such as methyl sulfanyl); sulfoamino; substituted or unsubstituted sulfonamide (such as benzo sulfonamide); substituted or unsubstituted sulfonate (such as chloro-phenyl sulfonate); lower alkylsulfinyl; phenylsulfinyl; phenyl-lower alkylsulfinyl; alkylphenylsulfinyl; lower alkanesulfonyl; phenylsulfonyl; phenyl-lower alkylsulfonyl; alkylphenylsulfonyl; halogen-lower alkylmercapto; halogen-lower alkylsulfonyl; such as especially trifluoromethane sulfonyl; phosphono (—P(═O)(OH)₂); hydroxy-lower alkoxy phosphoryl or di-lower alkoxyphosphoryl; substituted urea (such as 3 trifluoro-methyl-phenyl urea); alkyl carbamic acid ester or carbamates (such as ethyl-N-phenyl-carbamate) or —NR₄R₅, wherein R₄ and R₅ can be the same or different and are independently H; lower alkyl (e.g. methyl, ethyl or propyl); or R₄ and R₅ together with the N atom form a 3- to 8-membered heterocyclic ring containing 1-4 nitrogen, oxygen or sulfur atoms (e.g. piperazinyl, pyrazinyl, lower alkyl-piperazinyl, pyridyl, indolyl, thiophenyl, thiazolyl, n-methyl piperazinyl, benzothiophenyl, pyrrolidinyl, piperidino or imidazolinyl) where the heterocyclic ring may be substituted with any of the substituents defined herein.

Preferred substituents for the above groups include alkyl, phenyl, alkoxy, (such as methoxy), amino alkoxy, aminoethoxy, alkyl amino alkoxy, halo (such as fluoro, chloro or bromo).

Where the plural form is used for compounds, salts, pharmaceutical preparations, diseases and the like, this is intended to mean also a single compound, salt, or the like.

Salts are especially the pharmaceutically acceptable salts of compounds of formula (I) or (IA).

Such salts are formed, for example, as acid addition salts, preferably with organic or inorganic acids, from compounds of formula (I) or (IA) with a basic nitrogen atom, especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, trifluoroacetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethyl-amine or tri(2-hydroxyethyl)amine, or heterocyclic bases, for example N-ethyl-piperidine or N,N′-dimethylpiperazine.

When a basic group and an acid group are present in the same molecule, a compound of formula (I) or (IA) may also form internal salts.

For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred.

In view of the close relationship between the compounds in free form and those in the form of their salts, including those salts that can be used as intermediates, for example in the purification or identification of the compounds, tautomers or tautomeric mixtures and their salts, any reference to the compounds hereinbefore and hereinafter especially the compounds of the formula (I) or (IA), is to be understood as referring also to the corresponding tautomers of these compounds, especially of compounds of the formula (I) or (IA), tautomeric mixtures of these compounds, especially of compounds of the formula (I) or (IA), or salts of any of these, as appropriate and expedient and if not mentioned otherwise.

Where “a compound . . . , a tautomer thereof; or a salt thereof” or the like is mentioned, this means “a compound . . . , a tautomer thereof, or a salt of the compound or the tautomer”. Any asymmetric carbon atom may be present in the (R)-, (S)- or (R,S)-configuration, preferably in the (R)- or (S)-configuration. Substituents at a ring at atoms with saturated bonds may, if possible, be present in cis- (=Z-) or trans (=E-) form. The compounds may thus be present as mixtures of isomers or preferably as pure isomers, preferably as enantiomer-pure diastereomers or pure enantiomers.

### PREFERRED EMBODIMENTS ACCORDING TO THE INVENTION

In the following preferred embodiments, general expression can be replaced by the corresponding more specific definitions provided above and below, thus yielding stronger preferred embodiments of the invention.

Preferred is the USE of compounds of the formula (I) or (IA) or pharmaceutically acceptable salts thereof, for treatment of a proliferative disease.

Especially preferred is the USE of compounds of the formula (I) or (IA) or pharmaceutically acceptable salts thereof, for treatment of a proliferative disease where the disease to be treated is a disease depending on Hsp90 and/or an hsp90 client protein or a tumor which overexpresses Hsp90.

Also preferred is the USE of compounds of the formula (I) or (IA) or pharmaceutically acceptable salts thereof for the preparation of pharmaceutical preparations comprising compounds of formula (I) for the treatment of proliferative diseases, and optional pharmaceutically acceptable carriers.

The invention is also directed to compound of formula (IA).

In another embodiment, the invention further relates to a compound of formula (I) or (IA) and its use in the treatment of proliferative diseases or for the manufacture of pharmaceutical preparations, wherein:

R¹ is H; halo (such as chloro) lower alkyl (such as methyl, ethyl, propyl, isopropyl, butyl, isobutyl, t-butyl or propenyl); substituted lower alkyl (such as alkyl-lower alkyl or trifluoromethyl); cycloalkyl-alkyl (such as cyclopropyl-methyl or cyclopropyl-ethyl); arylalkyl (such as benzyl or phenylethyl) substituted arylalkyl (such as alkylbenzyl, fluorobenzyl, chlorobenzyl, bromobenzyl or alkyoxybenzyl);
R² is H, lower alkyl (such as methyl, ethyl, propyl, isopropyl, butyl, isobutyl, t-butyl or propenyl); substituted lower alkyl (such as alkyl-lower alkyl or trifluoromethyl); carboxy, —C—O-lower alkyl; SO₂-lower alkyl (such as SO₂-methyl); dialkylaminoalkylcarbamoyl (such as (2-dimethylamino-ethyl)methyl-carbamoyl); carbonyl or substituted carbonyl (such as piperaine-1-carbonyl, 4-methyl-piperaine-1-carbonyl and 4-ethyl-piperaine-1-carbonyl);
R³ is H, lower alkyl (such as methyl, ethyl, propyl, isopropyl, butyl, isobutyl, t-butyl or propenyl) or SO₂NH₂;
or pharmaceutically acceptable salts thereof.

Examples of compound within the scope of formula (I) include 1-(5-Chloro-2,4-dihydroxy-phenyl)-5-trifluoromethyl-1,3-dihydro-benzoimidazol-2-one; 1-(5-Ethyl-2,4-dihydroxy-phenyl)-5-trifluoromethyl-1,3-dihydro-benzoimidazol-2-one; 3-(5-Chloro-2,4-dihydroxy-phenyl)-2-oxo-2,3-dihydro-1H-benzoimidazole-5-sulfonic acid amide; 1-(5-Benzyl-2,4-dihydroxy-phenyl)-5-trifluoromethyl-1,3-dihydro-benzoimidazol-2-one; 1-(5-Benzyl-2,4-dihydroxy-phenyl)-5-methanesulfonyl-1,3-dihydro-benzoimidazol-2-one; 1-(5-Ethyl-2,4-dihydroxy-phenyl-5-methanesulfonyl-1,3-dihydro-benzoimidazol-2-one; 1-(5-Ethyl-2,4-dihydroxy-phenyl)-2-oxo-2,3-dihydro-1H-benzoimidazole-5-carboxylic acid; 1-(5-Ethyl-2,4-dihydroxy-phenyl)-2-oxo-2,3-dihydro-1H-benzoimidazole-5-carboxylic acid (2-dimethylamino-ethyl)-methyl-amide; 1-(5-Ethyl-2,4-dihydroxy-phenyl)-5-(piperazine-1-carbonyl)-1,3-dihydro-benzoimidazol-2-one; 1-(5-Ethyl-2,4-dihydroxy-phenyl)-5-(4-methyl-piperazine-1-carbonyl)-1,3-dihydro-benzoimidazol-2-one; 1-(5-Ethyl-2,4-dihydroxy-phenyl)-5-(4-ethyl-piperazine-1-carbonyl)-1,3-dihydro-benzoimidazol-2-one; 1-(5-Chloro-2-hydroxy-phenyl)-5-trifluoromethyl-1,3-dihydro-benzoimidazol-2-one; 1-(5-Chloro-2-hydroxy-phenyl)-5-trifluoromethyl-1,3-dihydro-benzoimidazol-2-one; and pharmaceutically acceptable salts thereof.

Where subsequently the term “USE” is mentioned, this includes any one or more of the following embodiments of the invention, respectively: the use in the treatment of proliferative diseases, especially those dependant on Hsp90 activity, the use for the manufacture of pharmaceutical compositions for use in the treatment of said diseases, pharmaceutical preparations comprising benzoimidazolone derivatives for the treatment of said diseases, and benzoimidazolone derivatives for use in the treatment of said diseases, as appropriate and expedient, if not stated otherwise. In particular, diseases to be treated and are thus preferred for USE of a compound of formula (I) or (IA) are selected from proliferative diseases, more especially diseases that depend on Hsp90 activity.

In a broader sense of the invention, a proliferative disease includes hyperproliferative conditions, such as leukemias, hyperplasias, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty. There is also a link between hsp90 and NFkB and this could lead to treatment for arthritis.

Very preferred is a method of treating a proliferative disease, preferably a benign or especially malignant tumor, more preferably carcinoma of the brain, kidney, liver, adrenal gland, bladder, breast, stomach (especially gastric tumors), ovaries, colon, rectum, prostate, pancreas, lung (especially SCLC), vagina, thyroid, sarcoma, glioblastomas, multiple myeloma or gastrointestinal cancer, especially colon carcinoma or colorectal adenoma, or a tumor of the neck and head, an epidermal hyperproliferation, especially psoriasis, prostate hyperplasia, a neoplasia, especially of epithelial character, preferably mammary carcinoma, or a leukemia. Most preferred are tumors that contain active and/or overexpressed hsp90 client proteins (e.g., ErbB-2, Braf, etc).

Compounds of formula (I) or (IA) are able to bring about the regression of tumors and to prevent the formation of tumor metastases and the growth of (also micro)metastases. In addition they can be used in epidermal hyperproliferation (e.g. psoriasis), in prostate hyperplasia, and in the treatment of neoplasias, especially of epithelial character, for example mammary carcinoma.

Compounds of formula (I) can also be used to treat or prevent fibrogenic disorders such as scleroderma (systemic sclerosis); diseases associated with protein aggregation and amyloid formation such as Huntington's disease; Inhibition of the replication of hepatitis C virus and treating hepatitis C virus; treating tumors associated with viral infection such as human papilloma virus; and inhibiting viruses dependent of heat-shock proteins.

The compounds of formula (I) or (IA) have valuable pharmacological properties and are useful in the treatment of proliferative diseases.

The inhibition of Hsp90 is measured using the procedure, with minor modifications, described in Schilb et al. Development and Implementation of a Highly Miniaturized Confocal 2D-FIDA-Based Analysis-Based High-Throughput Screening Assay to Search for Active Site Modulators of the Human Heat Shock Protein 90β, J of Biomolecular Screening, 2003 in press.

The procedure is repeated for different concentrations of test compound selected to cover the range of 0% to 100% inhibition and the concentration at which 50% inhibition of Hsp90 occurs (IC₅₀) for each compound is determined from concentration-inhibition curves in a conventional manner.

The compounds of the Examples hereinbelow have IC₅₀ values of the order of 50-1000 nM or less in the above mentioned FIDA assay, specifically ≦100 nM. Compounds with R⁴ being H have IC₅₀ values of the order of 10,000 nM or less in the above mentioned FIDA assay.

### Synthetic Procedure

The syntheses of the substituted anilines (i) & (ii) used as starting material are carried out as outlined in Scheme 1. 2,4-Dimethoxy-1-nitro-benzenes derivatives of formulas (iii) and (iv) are obtained by nitration of the corresponding alkyl-resorcinol (v) or 2,4-dimethoxy-acetophenone derivatives (vi). Nitration reagents can be either fuming nitric acid, cupric nitrate in acetic anhydride as disclosed in K. K. Weinhardt, Bioorg. Med. Chem. Lett. 6, 2687 1996, or tetramethylammonium nitrate/triflic anhydride in dichloromethane as disclosed in S. A. Shackelford, J. Org. Chem. 68, 267, 2003. Friedel-Crafts acylation reactions with 2,4-dimethoxy-1-nitro-benzene (vii) are performed preferably with cupric triflate as catalyst as disclosed in R. P. Singh, Tetrahedron 57, 241 2001. The 2,4-dimethoxy-5-substuted anilines (i) & (ii) are obtained by reduction of the corresponding nitro derivatives (iii) and (iv). The reduction is preferably done by hydrogenation over Pd(C) of either the 2,4-dimethoxy-5-nitro-alkylbenzene (iii) or the 2,4-dimethoxy-5-nitro-acetophenone derivatives (iv). Addition of hydrochloric acid for the simultaneous hydrogenation of the nitro and the keto group may be necessary for the full reduction of (iv). 2,4-Dimethoxy-1-alkyl-benzenes (v) are obtained by hydrogenation of the corresponding 2,4-dimethoxy-acetophenone (vi).

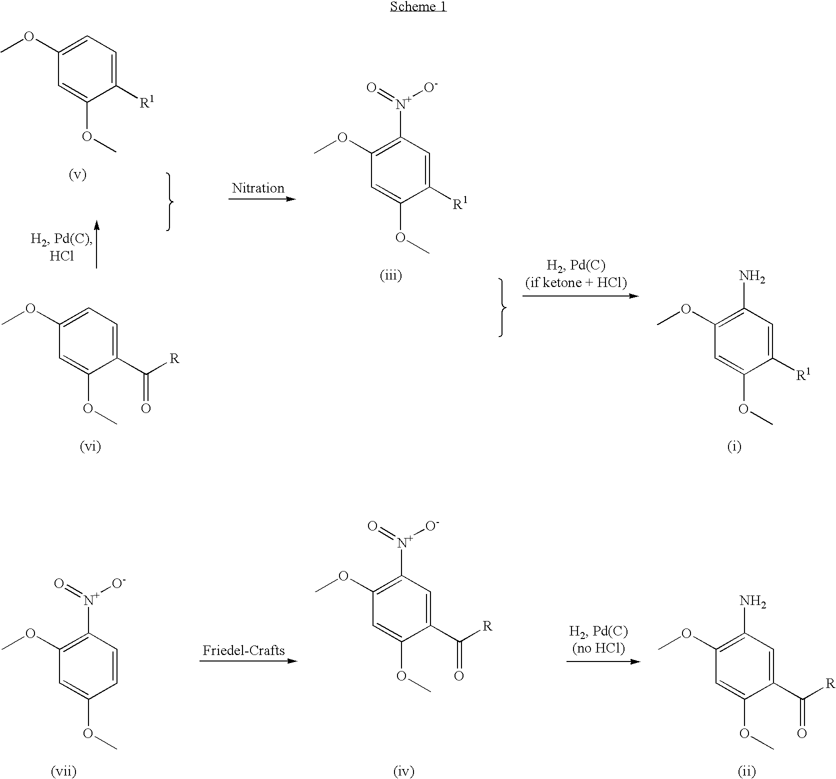

The syntheses of 1-(2,4-dihydroxy-phenyl)-1,3-dihydro-benzoimidazol-2-one derivatives of formula (I) or (IA) are carried out by using standard procedures as outlined in Scheme 2 below. A 5-substituted 2,4-dimethoxy aniline (i) and a substituted 2-fluoro-nitrobenzene (ix) are refluxed in THF (18-22 h) in presence of triethylamine to afford the nitrophenyl aniline adduct (x), which is isolated by crystallization or flash chromatography (55-96%). This SNAr reaction can also be catalyzed with a Pd phosphine complex: where residue X of (ix) is halogen, e.g., fluorine, the substitution reaction can be performed in DMF in the presence of triethylamine at 110° C. (1-17 h) using tetrakistriphenylphosphine Pd (0) as catalyst. If residue X is a trifluoro-methanesulfonic acid ester moiety (OTf), e.g., the substitution reaction can be performed in DMF in the presence of triethylamine at 110° C. (4 h) under Hartwig-Buchwald conditions, e.g., using Pd₂(dba)₃ [tris(dibenzylideneacetone)dipalladium(0)] as catalyst and rac-Binap [R(+)-2,2′-bis(diphenyl)phosphino)-1,1′-binaphthalen] as ligand.

The nitro group of compound (x) is then hydrogenated over Pd(C) or Pt(C) or Raney-Ni in either methanol or ethanol, the catalyst is filtered over Celite®, and the product (xi) is used without further purification in the next step. Thus, the crude N-Phenyl-benzene-1,2-diamine intermediate (xi) is treated with triphosgene or phosgene (20% sol. in toluene) in THF in presence of triethylamine. The reaction mixture is then stirred at RT or 50° C. (30 min. to 2 h) or refluxed after RT addition of phosgene (2 h) to afford the benzoimidazolone (xii), which is isolated by either crystallization or flash chromatography (44-85%). The dimethoxy deprotection of (xii) is done in dichloromethane with boron tribromide (1 M solution in dichloromethane), which is added at 0° C. After addition, the reaction mixture is stirred at RT (18 h to 48 h) to afford compounds of formula (viii) after flash chromatography. The demethylation can also be done in pyridinium hydrochloride at 170-190° C.

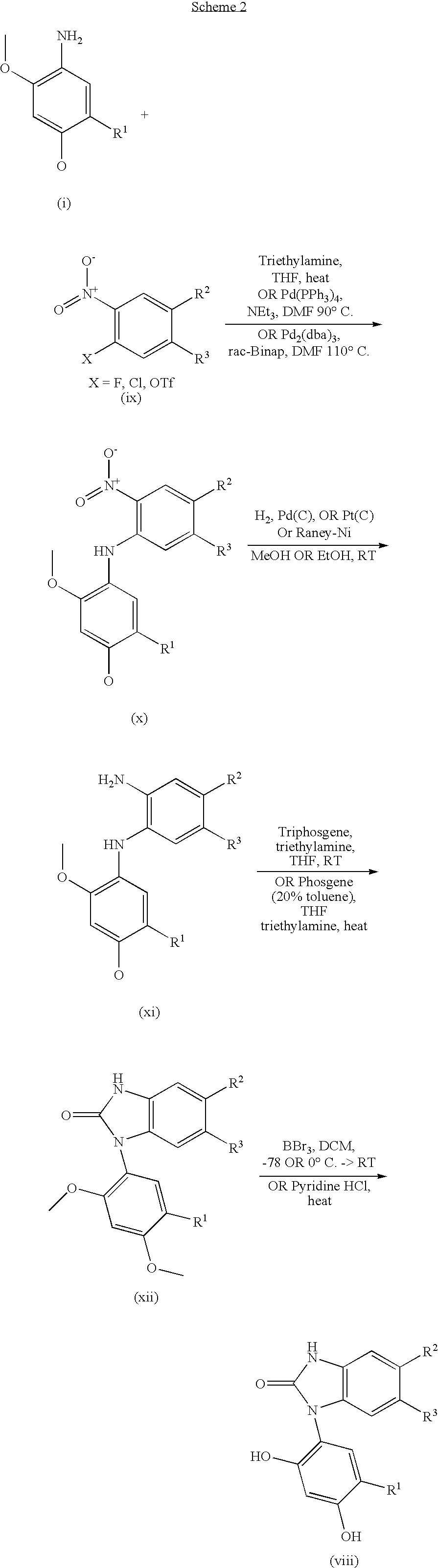

Derivatization of benzoimidazole-5-carboxylic acids is performed as shown in scheme 3 below:

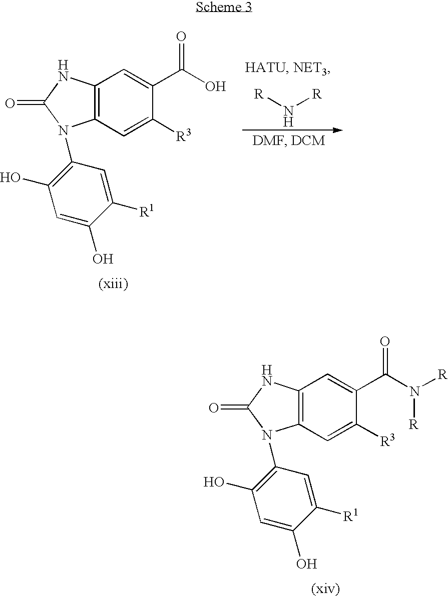

The deprotected benzoimidazole-5-carboxylic acid derivative (xiii) is condensed with a secondary amine using HATU [O-(7-azabenzotriazol-1-yl)-1,1,3,3-tetramethyluronium hexafluorophosphate], or other bond forming agents such as TBTU [2-(1H-Benzotriazole-1-yl)-1,1,3,3-tetramethyluronium tetrafluoroborate] or TPTU [2-(2-oxo-1(2H)-pyridyl-1,1,3,3-tetramethyluronium tetrafluoroborate], as coupling reagent as depicted in Scheme 3. Purification by MPLC afforded the TFA salt of compound (iv).

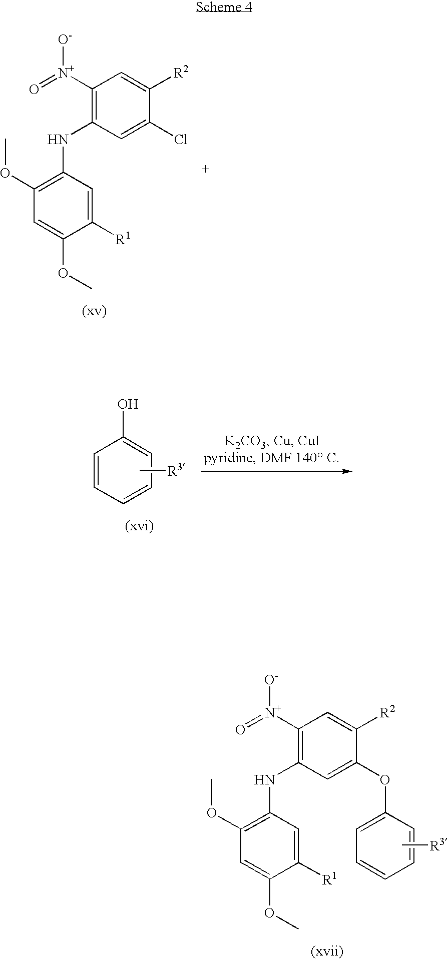

Compound (xv) obtained similarly to compound (x) can be further derivatized by S_{N}Ar at the chlorine position (R³) as depicted in Scheme 4. Thus, compound (xv) is reacted with a phenol derivative (xvi), analogously to the procedure as disclosed in R. F. Pellon Lomdom, Synth. Commun. 33, 921 2003, in presence of potassium carbonate, copper, copper iodide and traces of pyridine in DMF at 140° C. (1 h) to afford compound (xvii) (96%), which is a specific example of a compound of formula (x) that can be further modified as illustrated in Scheme 2.

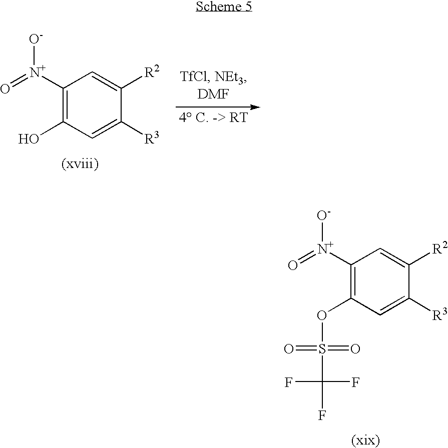

Compounds of formula (ix) can be obtained from commercial sources or can be synthesized by known procedures. For example (Scheme 5), an appropriate 2-nitro-phenol derivative (xviii) is transformed in the trifluoro-methanesulfonic acid 2-nitro-phenyl ester (xix) using trifluoromethanesulfonyl chloride in presence of triethylamine in DMF (86%). Thus, compound (xix), a specific example of a compound of formula (ix), can be used as starting material for the synthesis sequence depicted in Scheme 2.

Any of Schemes 2, 3, 4 or 5 above may further, if desired, involve transforming an obtainable compound of formula (I) or (IA) into a different compound of formula (I) or (IA), or into a salt thereof, or vice versa from a salt to free compound, in a conventional manner; and/or separating an obtainable mixture of isomers of compounds of formula (I) or (IA) into the individual isomers; where for all reactions mentioned functional groups in the starting materials that shall not take part in the reaction are, if required, present in protected form by readily removable protecting groups, and any protecting groups are subsequently removed.

The compounds in free or salt form can be obtained in the form of hydrates or solvates containing a solvent used for crystallization.

Salts of compound of formula (I) or (IA) can be prepared in a customary manner from the free compounds, and vice versa.

Mixtures of isomers obtainable according to the invention can be separated in a manner known per se into the individual Isomers; diastereoisomers can be separated, for example, by partitioning between polyphasic solvent mixtures, recrystallization and/or chromatographic separation, for example over silica gel or by e.g. medium pressure liquid chromatography over a reversed phase column, and racemates can be separated, for example, by the formation of salts with optically pure salt-forming reagents and separation of the mixture of diastereoisomers so obtainable, for example by means of fractional crystallization, or by chromatography over optically active column materials.

Intermediates and final products can be worked up and/or purified according to standard methods, e.g. using chromatographic methods, distribution methods, (re-) crystallization, and the like.

### General Process Conditions

The following applies in general to all processes mentioned hereinbefore and hereinafter, while reaction conditions specifically mentioned above or below are preferred:

All the above-mentioned process steps can be carried out under reaction conditions that are known per se, preferably those mentioned specifically, in the absence or, customarily, in the presence of solvents or diluents, preferably solvents or diluents that are inert towards the reagents used and dissolve them, in the absence or presence of catalysts, condensation or neutralizing agents, for example ion exchangers, such as caton exchangers, e.g. in the H⁺ form, depending on the nature of the reaction and/or of the reactants at reduced, normal or elevated temperature, for example in a temperature range of from about −100° C. to about 190° C., preferably from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers as described above.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofuran or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of those solvents, for example aqueous solutions, unless otherwise indicated in the description of the processes. Such solvent mixtures may also be used in working up, for example by chromatography or partitioning.

The compounds, including their salts, may also be obtained in the form of hydrates, or their crystals may, for example, include the solvent used for crystallization. Different crystalline forms may be present.

### Pharmaceutical Compositions

The invention relates also to pharmaceutical compositions comprising a compound of formula (I) or (IA), to their use in the therapeutic (in a broader aspect of the invention also prophylactic) treatment or a method of treatment of proliferative disease, especially the preferred diseases mentioned above, to the compounds for said use and to the preparation of pharmaceutical preparations, especially for said uses.

The pharmacologically acceptable compounds of the present invention may be used, for example, for the preparation of pharmaceutical compositions that comprise an effective amount of a compound of the formula (I) or (IA), or a pharmaceutically acceptable salt thereof, as active ingredient together or in admixture with a significant amount of one or more inorganic or organic, solid or liquid, pharmaceutically acceptable carriers.

The invention relates also to a pharmaceutical composition that is suitable for administration to a warm-blooded animal, especially a human (or to cells or cell lines derived from a warm-blooded animal, especially a human, e.g. lymphocytes), for the treatment or, in a broader aspect of the invention, prevention of (=prophylaxis against) a disease that responds to inhibition of Hsp90 activity, comprising an amount of a compound of formula (I) or (IA) or a pharmaceutically acceptable salt thereof, which is effective for said inhibition, especially the in, together with at least one pharmaceutically acceptable carrier.

The pharmaceutical compositions according to the invention are those for enteral, such as nasal, rectal or oral, or parenteral, such as intramuscular or intravenous, administration to warm-blooded animals (especially a human), that comprise an effective dose of the pharmacologically active ingredient, alone or together with a significant amount of a pharmaceutically acceptable carrier. The dose of the active ingredient depends on the species of warm-blooded animal, the body weight, the age and the individual condition, individual pharmacokinetic data, the disease to be treated and the mode of administration.

The invention relates also to a method of treatment for a disease that responds to inhibition of Hsp90; which comprises administering an (against the mentioned disease) prophylactically or especially therapeutically effective amount of a compound of formula (I) or (IA) according to the invention, especially to a warm-blooded animal, for example a human, that, on account of one of the mentioned diseases, requires such treatment.

The dose of a compound of the formula (I) or (IA) or a pharmaceutically acceptable salt thereof to be administered to warm-blooded animals, for example humans of approximately 70 kg body weight, is preferably from approximately 3 mg to approximately 10 g, more preferably from approximately 10 mg to approximately 1.5 g, most preferably from about 100 mg to about 1000 mg/person/day, divided preferably into 1-3 single doses which may, for example, be of the same size. Usually, children receive half of the adult dose.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragées, tablets or capsules.

The pharmaceutical compositions of the present invention are prepared in a manner known per se, for example by means of conventional dissolving, lyophilizing, mixing, granulating or confectioning processes.

Solutions of the active ingredient, and also suspensions, and especially isotonic aqueous solutions or suspensions, are preferably used, it being possible, for example in the case of lyophilized compositions that comprise the active ingredient alone or together with a carrier, for example mannitol, for such solutions or suspensions to be produced prior to use. The pharmaceutical compositions may be sterilized and/or may comprise excipients, for example preservatives, stabilizers, wetting and/or emulsifying agents, solubilizers, salts for regulating the osmotic pressure and/or buffers, and are prepared in a manner known per se, for example by means of conventional dissolving or lyophilizing processes. The said solutions or suspensions may comprise viscosity-increasing substances, such as sodium carboxymethylcellulose, carboxymethylcellulose, dextran, polyvinylpyrrolidone or gelatin.

Suspensions in oil comprise as the oil component the vegetable, synthetic or semi-synthetic oils customary for injection purposes. There may be mentioned as such especially liquid fatty acid esters that contain as the acid component a long-chained fatty acid having from 8-22, especially from 12-22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brasidic acid or linoleic acid, if desired with the addition of antioxidants, for example vitamin E, β-carotene or 3,5-di-tert-butyl-4-hydroxytoluene. The alcohol component of those fatty acid esters has a maximum of 6 carbon atoms and is a mono- or poly-hydroxy, for example a mono-, di- or tri-hydroxy, alcohol, for example methanol, ethanol, propanol, butanol or pentanol or the isomers thereof, but especially glycol and glycerol. The following examples of fatty acid esters are therefore to be mentioned: ethyl oleate, isopropyl myristate, isopropyl palmitate, “Labrafil M 2375” (polyoxyethylene glycerol trioleate, Gattefossé, Paris), “Miglyol 812” (triglyceride of saturated fatty acids with a chain length of C₈ to C₁₂, Hëls A G, Germany), but especially vegetable oils, such as cottonseed oil, almond oil, olive oil, castor oil, sesame oil, soybean oil and more especially groundnut oil.

The injection compositions are prepared in customary manner under sterile conditions; the same applies also to introducing the compositions into ampoules or vials and sealing the containers.

Pharmaceutical compositions for oral administration can be obtained by combining the active ingredient with solid carriers, if desired granulating a resulting mixture, and processing the mixture, if desired or necessary, after the addition of appropriate excipients, into tablets, dragée cores or capsules. It is also possible for them to be incorporated into plastics carriers that allow the active ingredients to diffuse or be released in measured amounts.

Suitable carriers are especially fillers, such as sugars, for example lactose, saccharose, mannitol or sorbitol, cellulose preparations and/or calcium phosphates, for example tricalcium phosphate or calcium hydrogen phosphate, and binders, such as starch pastes using for example corn, wheat, rice or potato starch, gelatin, tragacanth, methylcellulose, hydroxypropylmethylcellulose, sodium carboxymethylcellulose and/or polyvinylpyrrolidone, and/or, if desired, disintegrators, such as the above-mentioned starches, and/or carboxymethyl starch, crosslinked polyvinylpyrrolidone, agar, alginic acid or a salt thereof, such as sodium alginate. Excipients are especially flow conditioners and lubricants, for example silicic acid, talc, stearic acid or salts thereof, such as magnesium or calcium stearate, and/or polyethylene glycol. Dragée cores are provided with suitable, optionally enteric, coatings, there being used, inter alia, concentrated sugar solutions which may comprise gum arabic, talc, polyvinylpyrrolidone, polyethylene glycol and/or titanium dioxide, or coating solutions in suitable organic solvents, or, for the preparation of enteric coatings, solutions of suitable cellulose preparations, such as ethylcellulose phthalate or hydroxypropylmethylcellulose phthalate. Capsules are dry-filled capsules made of gelatin and soft sealed capsules made of gelatin and a plasticizer, such as glycerol or sorbitol. The dry-filled capsules may comprise the active ingredient in the form of granules, for example with fillers, such as lactose, binders, such as starches, and/or glidants, such as talc or magnesium stearate, and if desired with stabilizers. In soft capsules the active ingredient is preferably dissolved or suspended in suitable oily excipients, such as fatty oils, paraffin oil or liquid polyethylene glycols, it being possible also for stabilizers and/or antibacterial agents to be added. Dyes or pigments may be added to the tablets or dragée coatings or the capsule casings, for example for identification purposes or to indicate different doses of active ingredient

### Combinations

The compounds of the present invention may be administered alone or in combination with other anticancer agents, such as other antiproliferative agents and compounds that inhibit tumor angiogenesis, for example, the protease inhibitors; epidermal growth factor receptor kinase inhibitors; vascular endothelial growth factor receptor kinase inhibitors and the like; cytotoxic drugs, such as antimetabolites, like purine and pyrimidine analog antimetabolites; antineoplastic antimetabolites; antimitotic agents like microtubule stabilizing drugs and antimitotic alkaloids; platinum coordination complexes; anti-tumor antibiotics; alkylating agents, such as nitrogen mustards and nitrosoureas; endocrine agents, such as adrenocorticosteroids, androgens, anti-androgens, estrogens, anti-estrogens, aromatase inhibitors, gonadotropin-releasing hormone agonists and somatostatin analogues and compounds that target an enzyme or receptor that is overexpressed and/or otherwise involved a specific metabolic pathway that is upregulated in the tumor cell, for example ATP and GTP phosphodiesterase inhibitors, histone deacetylase inhibitors, bisphosphonates; protein kinase inhibitors, such as serine, threonine and tyrosine kinase inhibitors, for example, Abelson protein tryosine kinase and the various growth factors, their receptors and kinase inhibitors therefore, such as, epidermal growth factor receptor kinase inhibitors, vascular endothelial growth factor receptor kinase inhibitors, fibroblast growth factor inhibitors, insulin-like growth factor receptor inhibitors and platelet-derived growth factor receptor kinase inhibitors and the like; compounds targeting, decreasing or inhibiting the activity of the Axl receptor tyrosine kinase family, the c-Met receptor or the Kit/SCFR receptor tyrosine kinase; methionine aminopeptidase inhibitors; matrix metalloproteinase inhibitors (“(MMP”); agents used in the treatment of hematologic malignancies; Inhibitors of FMS-like tyrosine kinase receptors (Flt-3R); other Hsp90 inhibitors; antiproliferative antibodies such as trastuzumab (Herceptin™), Trastuzumab-DM1, erlotinib (Tarceva™), bevacizumab (Avastin™), rituximab (Rituxan®), PRO64553 (anti-CD40) and 2C4 Antibody; antibodies such as intact monoclonal antibodies, polyclonal antibodies; further anti-angiogenic compounds such as thalidomide and TNP470; compounds which target, decrease, or inhibit the activity of a protein or lipid phosphatase; compounds which induce cell differentiation processes; heparanase inhibitors; biological response modifiers; inhibitors of Ras oncogenic isoforms, e.g. farnesyl transferase inhibitors; telomerase inhibitors, methionine aminopeptidase inhibitors; proteasome inhibitors; and cyclooxygenase inhibitors, for example, cyclooxygenase-1 or -2 inhibitors. Also included are temozolomide, bengamides and m-Tor inhibitors.

The structure of the active agents identified by code nos., generic or trade names may be taken from the actual edition of the standard compendium “The Merck Index” or from databases, e.g. Patents International (e.g. IMS World Publications).

The above-mentioned compounds, which can be used in combination with a compound of the formula (I) or (IA), can be prepared and administered as described in the art such as in the documents cited above.

A compound of the formula (I) or (IA) may also be used to advantage in combination with known therapeutic processes, e.g., the administration of hormones or especially radiation.

A compound of formula (I) or (IA) may in particular be used as a radiosensitizer, especially for the treatment of tumors which exhibit poor sensitivity to radiotherapy.

The following examples serve to illustrate the invention without limiting the scope thereof:

### Syntheses General Conditions:

Flash chromatography is performed by using silica gel (Merck 60). MPLC is performed with reverse phase material (Merck LiChroprep® RP-18 using) using a Büchi pump system. For thin layer chromatography, precoated silica gel (Merck 60 F254) plates are used. Detection of the components is made by UV light (254 nm). HPLC analysis are performed with method (1) Agilent HP 1100 instrument, Nucleosil column 100-3 C18 HD 125×4, flow rate 1.0 mL/min gradient (a): 20% to 100% B in 7 min, gradient (b): 0% to 20% B in 3 min, then 20% B for 4 min. Or HPLC method (2) Thermo Finnigan Spectra SYSTEM, Chromolith Performance RP-18e 100×4.6, flow rate 2.0 mL/min; gradient: 2% to 100% B in 8 min, then 100% B for 2 min. For both HPLC method and MPLC solvent system is A=0.1% TFA in water, solvent B=0.1% TFA in acetonitrile. Electro spray mass spectra are obtained with a Fisons Instruments VG Platform II. ¹NMR measurements are performed on a Varian Gemini 400 spectrometer using tetraethylsilane as internal standard. Chemical shifts (8) are expressed in ppm downfield from tetraethylsilane and coupling constants (J) are expressed in Hertz.

Commercially available solvents and chemicals are used for all described syntheses.

### Example 1

The syntheses of the substituted anilines are carried out as follows: 2,4-Dimethoxy-1-nitro-benzenes derivatives of formulas (iii) and (iv) are obtained by nitration of the corresponding alkyl-resorcinol, formula (v), or 2,4-dimethoxy-acetophenone derivatives, formula (vi). Friedel-Crafts acylation reactions with 2,4-dimethoxy-1-nitro-benzene (vii) are performed with cupric triflate as catalyst. The 2,4-dimethoxy-5-substituted anilines are obtained by reduction of the corresponding nitro derivatives (iii) & (iv) by hydrogenation over, Pd(C) of either the 2,4-dimethoxy-5-nitro-alkylbenzene (iii) or the 2,4-dimethoxy-5-nitro-acetophenone derivatives (iv). Hydrochloric acid is added for the simultaneous hydrogenation of the nitro and the keto group. 2,4-Dimethoxy-1-alkyl-benzenes (v) are obtained by hydrogenation of the corresponding 2,4-dimethoxy-acetophenone (vi).

### Step 1.1: 5-Ethyl-2,4-dimethoxy-phenylamine

A solution of 1-ethyl-2,4-dimethoxy-5-nitro-benzene (Step 1.2) (6.1 g, 29 mmol) in ethanol (200 mL) is hydrogenated over Pd(C) (600 mg) for 1 h. The catalyst is filtered, the solvent is evaporated under reduced pressure, the residue crystallized from hexane to afford 5-ethyl-2,4-dimethoxy-phenylamine (Step 1.1) (4.1 g, 79%): HPLC method (2) t_{R}: 3.1, [M+H]⁺=182.

### Step 1.2: 1-Ethyl-2,4-dimethoxy-5-nitro-benzene

To a solution of tetramethylammonium nitrate (8.6 g, 66 mmol) in dichloromethane (300 mL) is added triflic anhydride (18.6 g, 66 mmol), and the solution is stirred for 1 h. After cooling the nitration solution to −70° C., a solution of 1-ethyl-2,4-dimethoxy-benzene (obtained from Pd(C) hydrogenation of 2,4-dimethoxyacetophenone) (10 g, 60 mmol) in dichloromethane (20 mL) is added drop wise. After 15 min the reaction mixture is allowed to warm to RT. The solution is extracted with a 10% hydrogen carbonate solution and dichloromethane, the combined organic phases are dried over sodium sulfate, the solvent is evaporated under reduced pressure and the residue is purified by column flash chromatography on silica gel (ethyl acetate/hexane 1:4) to afford 1-ethyl-2,4-dimethoxy-5-nitro-benzene (Step 1.2) (6.1 g, 48%): HPLC method (2) t_{R}: 5.2, [M+H]⁺=212.

### Example 2

Compound Example 2 is synthesized analogously to the procedures described in section Synthetic Procedure starting from 5-chloro-2,4-dimethoxy-phenylamine and 1-fluoro-2-nitro-4-trifluoromethyl-benzene.

### Example 3

To a solution of 1-(5-ethyl-2,4-dimethoxy-phenyl)-5-trifluoromethyl-1,3-dihydro-benzoimidazol-2-one (Step 3.1) (53 mg, 0145 mmol) in dichloromethane (6 mL) cooled to −70° C. is added a solution of boron tribromide (1N in dichloromethane, 1 mL). The solution is then allowed to reach RT, and is stirred for 24 h. The solution is treated with a 10% hydrogen carbonate solution and extracted with ethyl acetate. The combined organic phases are dried over sodium sulfate, the solvent is evaporated under reduced pressure and the residue is purified by column flash chromatography on silica gel (dichloromethane/methanol 19:1) to afford 1-(5-ethyl-2,4-dihydroxy-phenyl)-5-trifluoromethyl-1,3-dihydro-benzoimidazol-2-one (Example 3) (34 mg, 69%).

### Step 3.1:1-(5-Ethyl-2,4-dimethoxy-phenyl)-5-trifluoromethyl-1,3-dihydro-benzoimidazol-2-one

To a solution of N*1*-(5-Ethyl-2,4-dimethoxy-phenyl)-4-trifluoromethyl-benzene-1,2-diamine (Step 3.2) (98.7 mg, 0.29 mmol) and triethylamine (0.145 mL, 1 mmol) in THF (10 mL) at RT is added triphosgene (34 mg, 0.12 mmol). The reaction mixture is refluxed for 2 h, treated with a 10% hydrogen carbonate solution and extracted with ethyl acetate. The combined organic phases are dried over sodium sulfate, the solvent is evaporated under reduced pressure, and the residue is crystallized from diethyl ether/hexane to afford 1-(5-ethyl-2,4-dimethoxy-phenyl)-5-trifluoromethyl-1,3-dihydro-benzoimidazol-2-one (Step 3.1) (82 mg, 77%): HPLC method (2) t_{R}: 6.0, [M+H]⁺=367.

### Step 3.2: N*1*-(5-Ethyl-2,4-dimethoxy-phenyl)-4-trifluoromethyl-benzene-1,2-diamine 5-ethyl-2,4-dimethoxy-phenyl)-(2-nitro-4-trifluoromethyl-phenyl)-amine

A solution of (5-ethyl-2,4-dimethoxy-phenyl)-(2-nitro-4-trifluoromethyl-phenyl)-amine (Step 3.3) (375 mg, 1.1 mmol) in ethanol (50 mL) is hydrogenated over Raney-Nickel catalyst (6 mg) for 10 h. The catalyst is filtered over Celite®, the solvent is evaporated under reduced pressure, and the residue crystallized from hexane to afford N*1*-(5-ethyl-2,4-dimethoxy-phenyl)-4-trifluormethyl-benzene-1,2-diamine (Step 3.2) (153 mg, 41%): HPLC method (2) t_{R}: 6.0, [M+H]⁺=341.1.

### Step 3.3: (5-Ethyl-2,4-dimethoxy-phenyl)-(2-nitro-4-trifluoromethyl-phenyl)-amine

A solution of 5-ethyl-2,4-dimethoxy-phenylamine (Step 1.1) (380 mg, 2.1 mmol), 1-fluoro-2-nitro-4-trifluoromethyl-benzene (439 mg, 2.1 mmol), and triethylamine (0.23 mL, 2.1 mmol) in THF (10 mL) is heated at 80° C. for 18 h. The reaction mixture is cooled to RT, taken in diethyl ether, washed with water. The combined organic phases are dried over sodium sulfate, the solvent is evaporated under reduced pressure, and the residue crystallized from hexane to afford (5-ethyl-2,4-dimethoxy-phenyl)-(2-nitro-4-trifluoromethyl-phenyl)-amine (Step 3.3) (333 mg, 96%): HPLC method (2) t_{R}: 7.3, [M−H]⁻=369.

### Examples 4-7

Compounds Examples 4-7 are synthesized analogously to the procedure described for Example 3 using the appropriate corresponding starting materials.

### Example 8

1-(5-Ethyl-2,4-dimethoxy-phenyl)-2-oxo-2,3-dihydro-1H-benzoimidazole-5-carboxylic acid methyl ester (Step 8.1) (340 mg, 0.955 mmol) is stirred with pyridine hydrochloride (1.3 g) at 180° C. for 4 h under argon. The reaction mixture is taken up in aqueous citric acid solution (5%, 30 mL) and extracted with ethyl acetate (30 mL, 3×), The combined organic phases are dried over magnesium sulfate and concentrated under reduced pressure. Compound Example 8 is purified by crystallization from ethyl acetate/hexane to give a white solid (216 mg, 72%).

### Step 8.1:1-(5-Ethyl-2,4-dimethoxy-phenyl)-2-oxo-2,3-dihydro-1H-benzoimidazole-5-carboxylic acid methyl ester

Compound of Step 8.1 is synthesized analogously to the procedures described in section Synthetic Procedure starting from 5-ethyl-2,4-dimethoxy-phenylamine (Step 1.1) and 3-nitro-4-fluoro-benzoic acid methyl ester (Step 8.2).

### Step 8.2: 3-Nitro-4-fluoro-benzoic acid methyl ester

Compound of Step 8.2 is synthesized analogously to the procedure as disclosed in J. G. Avila-Zarraga, Synth. Commun. 31, 2177, 2001 by esterification of 3-nitro-4-fluoro-benzoic acid by means of methyl iodide/potassium hydroxide in DMSO. Purification is performed by flash chromatography (Ethyl acetate/hexane=1:1): Yield: 68%; EI-MS: M⁺=199, m. p.=58-62.5° C.

### Examples 9-12

Compounds of Examples 9-12 are synthesized from their carboxylic acid precursor (compound of Example 8) via amidation reaction with the corresponding amines: Compound of Example 8 (70 mg, 0.22 mmol), HATU [O-(7-azabenzotriazol-1-yl)-1,1,3,3-tetramethyluronium hexafluorophosphate] (89 mg, 0.23 mmol), triethylamine (0.04 mL) dissolved in DMF/dichloromethane (2:3; 3.3 mL) and stirred for 15 min at RT. After adding the corresponding amine (0.7 mmol) dissolved in dichloromethane (1 mL), the reaction mixture is stirred over night. After evaporating the solvent, the crude mixtures are purified by preparative reversed phase chromatography using Buchi (Flawil) MPLC system (B-688 pump, B-687 gradient former) and 2.6×26 cm column loaded with Lichroprep RP18 (15-25 μm) from E. Merck (Darmstadt): 0->24% B in 10 min/remaining at 24% B for 15 min/->100% B in 10 min. The corresponding fractions are concentrated under reduced pressure and then further lyophilized. Yields of compounds of Examples 9-12: 34, 83, 70, 60%, respectively.

### Example 13

Compound of Example 13 is synthesized analogously to the procedures described in section Synthetic Procedure starting from 5-ethyl-2,4-dimethoxy-phenylamine (Step 1.1) and trifluoro-methanesulfonic acid 4-tert-butyl-2-nitro-phenyl ester (Step 13.1).

### Step 13.1: Trifluoro-methanesulfonic acid 4-tert-butyl-2-nitro-phenyl ester

To a solution of 4-tert.-butyl-2-nitrophenol (0.893 mL, 5.12 mmol) dissolved in DMF (10 mL), triethylamine (0.785 mL, 5.63 mmol) was added at 4° C. After stirring for 10 min, trifluoromethanesulfonyl chloride (1.138 mL, 1.075 mmol) is added and the reaction mixture is stirred over night at RT. After adding concentrated ammonium chloride solution (70 mL), the resulting reaction solution is extracted with ethyl acetate (80 mL, 2×). The combined organic solutions are dried over magnesium sulfate, concentrated under reduced pressure, and flash chromatographed (silica gel, 3.6×18 cm, ethyl acetate/hexane=1:9) to give trifluoromethanesulfonic acid 4-tert-butyl-2-nitro-phenyl ester (Step 13.1) as colorless solid (1.44 g, 86%): [M+H]⁺=328.0, HPLC method (1) gradient (a) t_{R}: 7.68 min.

### Example 14a&b

### Modification Scheme 4

### 1-(5-Ethyl-2,4-dimethoxy-phenyl)-6-phenoxy-5-trifluoromethyl-1,3-dihydrobenzoimidazol-2-one (Step 14a.1)

N*2*-(5-Ethyl-2,4-dimethoxy-phenyl)-4-phenoxy-5-trifluoromethyl-benzene-1,2-diamine (Step 14a.2) (93 mg, 0.215 mmol), phosgen (20% in toluene, 0.20 ml, 0.40 mmol), and NEt₃ (0.13 mL) are dissolved in THF (9 mL) at 0° C. After stirring at 55° C. for 2.5 h under Ar, the solvent is evaporated and the resulting crude residue is purified by recrystallization from CH₂Cl₂/hexane and further preparative TLC: 2 20×20 cm silica gel plates (AcOEt/hexane=1:1) to give white crystals: 75 mg (0.164 mmol, 76%), [M+H]⁺=459.1, m.p. >240° C., R^{F} (AcOEt/hexane=1:1)=0.40.

### N-2′-(5-Ethyl-2,4-dimethoxy-phenyl)phenoxy-5-trifluoromethyl-benzene-1,2-diamine (Step 14a.2)

Is generated from 5-ethyl-2,4-dimethoxy-phenyl)-(2-nitro-5-phenoxy-4-trifluoromethyl-phenyl)-amine (Step 14a.3) by hydrogenation (Pt/C, 10%, ethanol, 30 min): white solid, quantitative yield, [M+H]⁺=433.0, (AcOEt/hexane=1:1)=0.50. ¹H-NMR (CDCl₃, 400 MHz): 7.31 (t, 8.5 Hz, 1H, phenyl), 7.11 (s, 1H), 7.04 (t, 8.5 Hz, 2H, phenyl), 6.97 (d, 8.5 Hz, 2H, phenyl), 6.8016.71/6.49 (s/s/s, 1H/1H/1H), 3.84/3.82 (s/s, 3H/3H, OMe/OMe), 2.45 (q, 8.0 Hz, 2H, CH₂-ethyl), 1.02 (t, 8.0 Hz, 3H, CH₃-ethyl).

### 5-Ethyl-2,4-dimethoxy-phenyl)-(2-nitro-5-phenoxy-4-trifluoromethyl-phenyl)-amine (Step 14a.3)

Is synthesized in analogy of the procedure described by R. F. Pellon Lomdom and M. L. Decampo Palacios (Synth. Commun. 2003, 33 (6), 921-926) by stirring (5-chloro-2-nitro-4trifluoromethyl-phenyl)-(5-ethyl-2,4-dimethoxy-phenyl)-amine (Step 14a.4) (100 mg, 0.247 mmol) and phenol (34.4 mg, 0.366 mmol): dissolved in DMF (7 mL) in the presence of K₂CO₃ (450 mg) and of catalytical amounts of pyridine, Cu, and CuI at 140° C. for 60 min. After filtration of the reaction mixture, the solvent is evaporated under reduced pressure. Compound of Step 14a.3 is isolated by precipitaion from CH₂Cl₂/hexane as white solid (110 mg, 0.238 mmol, 96%), [M+H]⁺=463.1, R^{F} (CH₂Cl₂/hexane=1:1)=0.32.

### (5-Chloro-2-nitro-4-trifluoromethyl-phenyl-(5-ethyl-2,4-dimethoxy-phenyl)-amine (Step 14a.4)

5-Ethyl-2,4-dimethoxy-phenylamine (Step 1.1) (600 mg, 3.32 mmol), 1,5-dichloro-2-nitro-4-trifluoromethyl-benzene (862 mg, 3.32 mmol), NEt₃ (0.4 mL), and tetrakistriphenylphosphine palladium (6 mg) dissolved in DMF (10 mL) are stirred at 90° C. for 1 h. After evaporation of the solvent, the residue is purified by flash chromatography (6.5×16.5 cm, silica gel, CH₂Cl₂/hexane=1:1) to give compound of Step 14a.4 as orange solid: 1.24 g (307 mmol, 92.5%), [M−H]⁺=403.0/405.0, (AcOEt/hexane=1:1)=0.37, m.p.=161-162° C.

### 5-Acetyl-1-(5-ethyl-2,4-dihydroxy-phenyl)-1,3-dihydro-benzoimidazol-2-one (14b)

A mixture of 5-acetyl-1-(5-ethyl-2,4-dimethoxy-phenyl)-1,3-dihydro-benzoimidazol-2-one (Step 14b.1) (0.1 g, 0.294 mmol) and pyridine hydrochloride (173 mg, 1.47 mmol) are stirred at 180° C. for 3 h. After cooling down to r.t., H₂O (5 mL) is added and the resulting solution is acidified by means of aqueous citric acid solution (5%, 3 mL). The thus resulting solution is extracted with AcOEt (20 mL, 3×). The combined organic layers are dried (MgSO₄) and the solvent is evaporated under reduced pressure. The product is isolated by flash chromatography using a Combi Flash Companion from Isco Teledyne (12 g RediSept silica gel column, CH₂Cl₂/MeOH=95:5): 25 mg (0.080 mmol, 27%), [M+H]⁺=313.1, HPLC: t_{R}=4.17 min (method B, gradient b), ¹H-NMR (DMSO-d₆, 400 MHz): 11.18 (s, 1H, NH), 9.56/9.48 (s/s, 1H/1H, OH/OH), 7.69 (d, 8.5 Hz, 1H), 7.54 (s, 1H), 6.92 (s, 1H), 6.64 (d, 8.5 Hz, 1H), 6.55 (s, 1H), 2.50 (s, 3H, acetyl), 2.44 (q, 7.5 Hz, 2H, CH₂-ethyl), 1.10 (t, 7.5 Hz, 3H, CH₃-ethyl).

5-Acetyl-1-(5-ethyl-2,4-dimethoxy-phenyl)-1,3-dihydro-benzoimidazol-2-one (Step 14b.1) 1-[3-Amino-4(5-ethyl-2,4-dimethoxy-phenylamino)-phenyl]-ethanone (Step 14b.2) (1.31, 4.17 mmol), phosgen (20% in toluene, 3.09 ml, 4.16 mmol), and NEt₃ (2.09 ml, 15 mmol) are dissolved in THF (60 mL) at r.t. After stirring at 66° C. for 1 h under Ar, the solvent is evaporated and the resulting crude residue is recrystallized from AcOEt/hexane and to give beige crystals: 922 mg (2.71 mmol, 65%), [M+H]⁺=341.1, HPLC: t_{R}=8.04 min (method B, gradient b).

### 1-[3-Amino-4-(5-ethyl-2,4-dimethoxy-phenylamino)-phenyl]-ethanone (Step 14b.2)

1-[4(5-Ethyl-2,4-dimethoxy-phenylamino)-3-nitro-phenyl]-ethanone (Step 14b.3) (1.67 g, 4.85 mmol) dissolved in MeOH/THF (3:1, 40 mL) is hydrogenated in the presence of Raney-Ni (0.4 g) during 12 h at r.t. The product is isolated by flash chromatography using a Combi Flash Companion from Isco Teledyne (120 g RediSept silica gel column, CH₂Cl₂/MeOH 98:2): 1.32 g (4.17 mol, 87%), [M+H]⁺=315.2, HPLC: t_{R}=4.97 min (method B, gradient b).

### 1-[4(5-Ethyl-2,4-dimethoxy-phenylamino)-3-nitro-phenyl]-ethanone (Step 14b.3)

5-Ethyl-2,4-dimethoxy-phenylamine (Step 1.1) (990 mg, 5.46 mmol), 1-(4-Fluoro-3-nitro-phenyl)-ethanone (1 g, 5.46 mmol), NEt₃ (1.06 mL, 7.64 mmol), and tetrakistriphenylphosphine palladium (12 mg) dissolved in DMF (10 mL) are stirred at 90° C. for 16 h. When cooling the reaction mixture by means of an ice bath, compound of Step 14b.3 is precipitating as white solid, which is washed by MeOH and dried under vacuum at 50° C. for 12 h: 1.69 g (4.91 mmol, 90%), [M+H]⁺=345.0, HPLC: t_{R}=7.41 min (method B, gradient b).

| | | HPLC | MS |
|---|---|---|---|
| EX | Name | t_{R} [min] | [M + H]⁺ |
| 2 | 1-(5-Chloro-2,4-dihydroxy-phenyl)-5- | 6.77¹ | 345/347 |
| | trifluoromethyl-1,3-dihydro-benzoimidazol-2-one | | |
| 3 | 1-(5-Ethyl-2,4-dihydroxy-phenyl)-5- | 4.77² | 339 |
| | trifluoromethyl-1,3-dihydro-benzoimidazol-2-one | | |
| 4 | 3-(5-Chloro-2,4-dihydroxy-phenyl)-2-oxo-2,3- | 2.76² | 354 (−) |
| | dihydro-1H-benzoimidazole-5-sulfonic acid | | |
| | amide | | |
| 5 | 1-(5-Benzyl-2,4-dihydroxy-phenyl)-5- | 5.10² | 401 |
| | trifluoromethyl-1,3-dihydro-benzoimidazol-2-one | | |
| 6 | 1-(5-Benzyl-2,4-dihydroxy-phenyl)-5- | 3.88² | 410.9 |
| | methanesulfonyl-1,3-dihydro-benzoimidazol-2- | | |
| | one | | |
| 7 | 1-(5-Ethyl-2,4-dihydroxy-phenyl)-5- | 3.40² | 349 |
| | methanesulfonyl-1,3-dihydro-benzoimidazol-2- | | |
| | one | | |
| 8 | 1-(5-Ethyl-2,4-dihydroxy-phenyl)-2-oxo-2,3- | 0.19³ | 315.1 |
| | dihydro-1H-benzoimidazole-5-carboxylic acid | | |
| 9 | 1-(5-Ethyl-2,4-dihydroxy-phenyl)-2-oxo-2,3- | 0.35⁴ | 399 |
| | dihydro-1H-benzoimidazole-5-carboxylic acid (2- | | |
| | dimethylamino-ethyl)-methyl-amide | | |
| 10 | 1-(5-Ethyl-2,4-dihydroxy-phenyl)-5-(piperazine-1- | 6.20⁵ | 383 |
| | carbonyl)-1,3-dihydro-benzoimidazol-2-one | | |
| | (TFA) | | |
| 11 | 1-(5-Ethyl-2,4-dihydroxy-phenyl)-5-(4-methyl- | 6.43⁵ | 397 |
| | piperazine-1-carbonyl)-1,3-dihydro- | | |
| | benzoimidazol-2-one (TFA) | | |
| 12 | 1-(5-Ethyl-2,4-dihydroxy-phenyl)-5-(4-ethyl- | 6.82⁵ | 411 |
| | piperazine-1-carbonyl)-1,3-dihydro- | | |
| | benzoimidazol-2-one (TFA) | | |
| 13 | 5-tert-Butyl-1-(5-ethyl-2,4-dihydroxy-phenyl)-1,3- | 6.82¹ | 327.1 |
| | dihydro-benzoimidazol-2-one | | |
| 14a | 1-(5-Ethyl-2,4-dihydroxy-phenyl)-6-phenoxy-5- | 6.14¹ | 431.1 |
| | trifluoromethyl-1,3-dihydro-benzoimidazol-2-one | | |
| 14b | 5-Acetyl-1-(5-ethyl-2,4-dihydroxy-phenyl)-1,3-dihydro- | | |
| | benzoimidazol-2-one | | |

¹HPLC method (1), gradient (a)
²HPLC method (2)
³TLC Silica gel, MeOH/DCM 15:85
⁴TLC Silica gel, MeOH/DCM/NH₃ 20:80:0.5
⁵HPLC method (1), gradient (b)

### Example 15

### Tablets 1 Comprising Compounds of the Formula (I)

Tablets, comprising, as active ingredient, 50 mg of any one of the compounds of formula (I) mentioned in the preceding Examples 2-14 of the following composition are prepared using routine methods:

| Composition: | |
|---|---|
| Active Ingredient | 50 mg |
| Wheat starch | 60 mg |
| Lactose | 50 mg |
| Colloidal silica | 5 mg |
| Talcum | 9 mg |
| Magnesium stearate | 1 mg |
| | 175 mg |

Manufacture: The active ingredient is combined with part of the wheat starch, the lactose and the colloidal silica and the mixture pressed through a sieve. A further part of the wheat starch is mixed with the 5-fold amount of water on a water bath to form a paste and the mixture made first is kneaded with this paste until a weakly plastic mass is formed.

The dry granules are pressed through a sieve having a mesh size of 3 mm, mixed with a pre-sieved mixture (1 mm sieve) of the remaining corn starch, magnesium stearate and talcum and compressed to form slightly biconvex tablets.

### Example 16

### Tablets 2 Comprising Compounds of the Formula (I)

Tablets, comprising, as active ingredient, 100 mg of any one of the compounds of formula (I) of Examples 2-14 are prepared with the following composition, following standard procedures:

| Composition: | |
|---|---|
| Active Ingredient | 100 mg |
| Crystalline lactose | 240 mg |
| Avicel | 80 mg |
| PVPPXL | 20 mg |
| Aerosil | 2 mg |
| Magnesium stearate | 5 mg |
| | 447 mg |

Manufacture: The active ingredient is mixed with the carrier materials and compressed by means of a tabletting machine (Korsch EKO, Stempeldurchmesser 10 mm).

### Example 16

### Capsules

Capsules, comprising, as active ingredient, 100 mg of any one of the compounds of formula (I) given in Examples 2-14, of the following composition are prepared according to standard procedures:

| Composition: | |
|---|---|
| Active Ingredient | 100 mg |
| Avicel | 200 mg |
| PVPPXL | 15 mg |
| Aerosil | 2 mg |
| Magnesium stearate | 1.5 mg |
| | 318.5 mg |

Manufacturing is done by mixing the components and filling them into hard gelatine capsules, size 1.

What is claimed is:

1. Use of compounds of the formula (I), 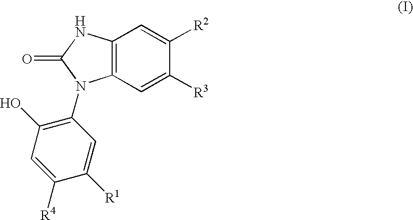
wherein:
R¹ is H, halo, substituted or unsubstituted lower alkyl;
R² is H, halo, substituted or unsubstituted lower alkyl, carboxy, COR⁵, SO₂R⁵, CX₂R⁵, CXHR⁵, CH₂R⁵, CHR⁵R⁶, C R⁵(R⁶)₂ C(R⁵)₂R⁶;
R³ is H, substituted or unsubstituted lower alkyl, halo, —SO₂NH₂ or 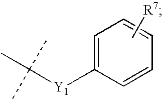
R⁴ is H or hydroxy;
R⁵ is lower alkyl; —(CH₂)ₙ—NR⁶₂; —YR⁶; —Y(CH₂)ₘ—NR⁶₂; 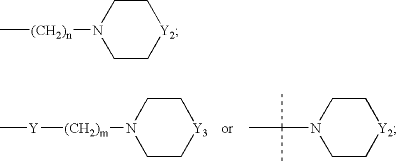
n is 1 or 2;
m is 2 or 3;
X is halo;
Y₁ is alkylene, O, S or N;
Y₂ and Y₃ are each independently methylene, O or NR′;
R⁶ is H, lower alkyl, cycloalkyl, heterocycl, fused cycloalkyl, fused heterocycl or NR⁹R¹⁰ together form a heterocyclic ring with the N atom, form a 3- to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl); cycloalkyl as defined above, especially C₃-C₆cycloalkyl, lower alkanoyl (preferably as single amino substituent or in combination with another of the non-acyl moiety just mentioned) and benzoyl or phenyl-lower alkanoyl (preferably as single amino substituent or in combination with another of the non-acyl moiety just mentioned), cyano, cyano-lower alkyl, such as cyanomethyl, amidino, N-hydroxyamidino, amidino-lower alkyl, such as -methyl, or N-hydroxyamidino-lower alkyl, such as -methyl;
R⁷ is lower alkyl, halo, lower alkoxy, —Y₁-(CH₂)ₚ—N(R⁸)(H);
p is 1-3;
R⁸ is H or lower alkyl;
or pharmaceutically acceptable salts thereof, for treatment of a proliferative disease.

2. A use according to claim 1 wherein the disease to be treated is a disease depending on Hsp90 and/or a hsp90 client protein or a tumor which overexpresses Hsp90.

3. Use of compounds of the formula (I) according to claim 1, or pharmaceutically acceptable salts thereof for the manufacture of pharmaceutical compositions for use in the treatment of proliferative diseases.

4. A compound according to formula (I): 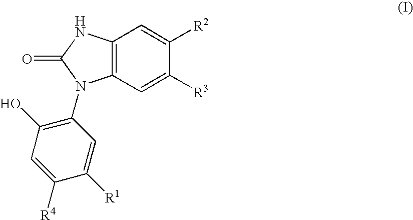
wherein:
R¹ is H, halo, substituted or unsubstituted lower alkyl;
R² is H, halo, substituted or unsubstituted lower alkyl, carboxy, COR⁵, SO₂R⁵, CX₂R⁵, CXHR⁵, CH₂R⁵, CHR⁵R⁶, CR⁵(R⁶)₂C(R⁵)₂R⁶;
R³ is H, substituted or unsubstituted lower alkyl, halo, —SO₂NH₂ or 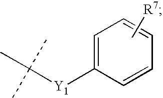
R⁴ is OH;
R⁵ is lower alkyl; —(CH₂)ₙ—NR⁶₂; —YR⁶; —Y(CH₂)ₘ—NR⁶₂; 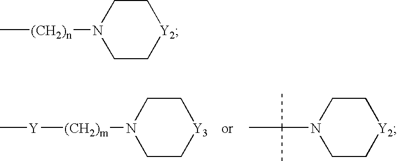
n is 1 or 2;
m is 2 or 3;
X is halo;
Y₁ is alkylene, O, S or N;
Y₂ and Y₃ are each independently methylene, O or NR′;
R⁶ is H, lower alkyl, cycloalkyl, heterocycl, fused cycloalkyl, fused heterocycl or NR⁹R¹⁰ together form a heterocyclic ring with the N atom, form a 3- to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl); cycloalkyl as defined above, especially C₃-C₆cycloalkyl, lower alkanoyl (preferably as single amino substituent or in combination with another of the non-acyl moiety just mentioned) and benzoyl or phenyl-lower alkanoyl (preferably as single amino substituent or in combination with another of the non-acyl moiety just mentioned), cyano, cyano-lower alkyl, such as cyanomethyl, amidino, N-hydroxyamidino, amidino-lower alkyl, such as -methyl, or N-hydroxyamidino-lower alkyl, such as -methyl;
R⁷ is lower alkyl, halo, lower alkoxy, —Y₁—(CH₂)ₚ—N(R⁸)(H);
p is 1-3;
R⁷ is H or lower alkyl;
or pharmaceutically acceptable salts thereof.

5. A use according to claim 1 wherein:
R¹ is H; halo (such as chloro) lower alkyl (such as methyl, ethyl, propyl, isopropyl, butyl, isobutyl, t-butyl or propenyl); substituted lower alkyl (such as alkyl-lower alkyl or trifluoromethyl); cycloalkyl-alkyl (such as cyclopropyl-methyl or cyclopropyl-ethyl); arylalkyl (such as benzyl or phenylethyl) substituted arylalkyl (such as alkylbenzyl, fluorobenzyl, chlorobenzyl, bromobenzyl or alkyoxybenzyl);
R² is H, lower alkyl (such as methyl, ethyl, propyl, isopropyl, butyl, isobutyl, t-butyl or propenyl); substituted lower alkyl (such as alkyl-lower alkyl or trifluoromethyl); carboxy, —C—O-lower alkyl; SO₂-lower alkyl (such as SO₂-methyl); dialkylaminoalkylcarbamoyl (such as (2-dimethylamino-ethyl)methyl-carbamoyl); carbonyl or substituted carbonyl (such as piperaine-1-carbonyl, 4-methyl-piperaine-1-carbonyl and 4-ethyl-piperaine-1-carbonyl);
R³ is H, lower alkyl (such as methyl, ethyl, propyl, isopropyl, butyl, isobutyl, t-butyl or propenyl) or SO₂NH₂;
or pharmaceutically acceptable salts thereof.

6. Method of treating a proliferative disease comprising administering a compound according to claim (I): 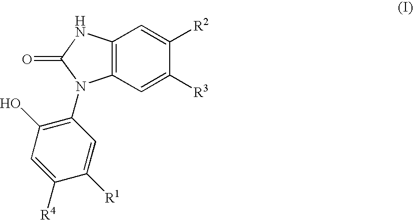
wherein:
R¹ is H, halo, substituted or unsubstituted lower alkyl;
R² is H, halo, substituted or unsubstituted lower alkyl, carboxy, COR⁵, SO₂R⁵, CX₂R⁵, CXHR⁵, CH₂R⁵, CHR⁵R⁶, C R⁵(R⁵)₂, C(R⁵)₂R⁶;
R³ is H, substituted or unsubstituted lower alkyl, halo, —SO₂NH₂ or 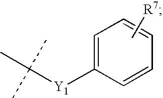
R⁴ is H or hydroxy;
R⁵ is lower alkyl; —(CH₂)ₙ—NR⁶₂; —YR⁶; —Y(CH₂)ₘ—NR⁶₂; 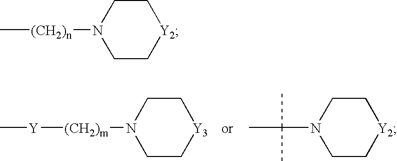
n is 1 or 2;
m is 2 or 3;
X is halo;
Y₁ is alkylene, O, S or N;
Y₂ and Y₃ are each independently methylene, O or NR′;
R⁶ is H, lower alkyl, cycloalkyl, heterocycl, fused cycloalkyl, fused heterocycl or NR⁹R¹⁰ together form a heterocyclic ring with the N atom, form a 3- to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl); cycloalkyl as defined above, especially C₃-C₆cycloalkyl, lower alkanoyl (preferably as single amino subsUtuent or in combination with another of the non-acyl moiety just mentioned) and benzoyl or phenyl-lower alkanoyl (preferably as single amino substituent or in combination with another of the non-acyl moiety just mentioned), cyano, cyano-lower alkyl, such as cyanomethyl, amidino, N-hydroxyamidino, amidino-lower alkyl, such as -methyl, or N-hydroxyamidino-lower alkyl, such as -methyl;
R⁷ is lower alkyl, halo, lower alkoxy, —Y₁—(CH₂)ᵣ—N(R⁸)(H);
p is 1-3;
R⁸ is H or lower alkyl;
or pharmaceutically acceptable salts thereof.

7. A method according to claim 6, wherein the proliferative disease is a benign or malignant tumor, a carcinoma of the brain, kidney, liver, adrenal gland, bladder, breast, stomach, gastric tumors, ovaries, colon, rectum, prostate, pancreas, lung, vagina, thyroid, sarcoma, glioblastomas, multiple myeloma or gastrointestinal cancer, colon carcinoma or colorectal adenoma, or a tumor of the neck and head, an epidermal hyperproliferation, prostate hyperplasia, a neoplasia, or a leukemia.

8. A pharmaceutical composition comprising a compound according to claim 4.

9. A pharmaceutical composition comprising a compound according to claim 4 and an acceptable pharmaceutical carrier.

10. A compound according to claim 4 selected from the group consisting of:
1-(5-Chloro-2,4-dihydroxy-phenyl)-5-trifluoromethyl-1,3-dihydro-benzoimidazol-2-one;
1-(5-Ethyl-2,4-dihydroxy-phenyl)-5-trifluoromethyl-1,3-dihydro-benzoimidazol-2-one;
3-(5-Chloro-2,4-dihydroxy-phenyl)-2-oxo-2,3-dihydro-1H-benzoimidazole-5-sulfonic acid amide;
1-(5-Benzyl-2,4-dihydroxy-phenyl)-5-trifluoromethyl-1,3-dihydro-benzoimidazol-2-one;
1-(5-Benzyl-2,4-dihydroxy-phenyl)-5-methanesulfonyl-1,3-dihydro-benzoimidazol-2-one;
1-(5-Ethyl-2,4-dihydroxy-phenyl)-5-methanesulfonyl-1,3-dihydro-benzoimidazol-2-one;
1-(5-Ethyl-2,4-dihydroxy-phenyl)-2-oxo-2,3-dihydro-1H-benzoimidazole-5-carboxylic acid;
1-(5-Ethyl-2,4-dihydroxy-phenyl)-2-oxo-2,3-dihydro-1H-benzoimidazole-5-carboxylic acid (2-dimethylamino-ethyl)-methyl-amide;
1-(5-Ethyl-2,4-dihydroxy-phenyl)-5-(piperazine-1-carbonyl-1,3-dihydro-benzoimidazol-2-one;
1-(5-Ethyl-2,4-dihydroxy-phenyl)-5-(4-methyl-piperazine-1-carbonyl)1,3-dihydro-benzoimidazol-2-one;
1-(5-Ethyl-2,4-dihydroxy-phenyl)-5-(4-ethyl-piperazine-1-carbonyl)-1,3-dihydro-benzoimidazol-2-one;
5-Acetyl-1-(5-ethyl-2,4-dihydroxy-phenyl)-1,3-dihydro-benzoimidazol-2-one;
and pharmaceutically acceptable salts thereof.

11. A process to prepare a compound according to claim 4 comprising deprotecting the demethyoxy of a benxoimidazolone derivative.
